(19) United States
(12) Patent Application Publication
(10) Pub. No.: US 2025/0127952 A1
(43) Pub. Date: Apr. 24, 2025

(54) POLYMERIC MATRIX FOR HAEMOSTATIC APPLICATION AND THERAPEUTIC BANDAGE THEREOF

(71) Applicant: INDIAN INSTITUTE OF TECHNOLOGY KANPUR, Kanpur, Uttar Pradesh (IN)

(72) Inventors: Ashok KUMAR, Kanpur, Uttar Pradesh (IN); Syed Muntazir ANDRABI, Kanpur, Uttar Pradesh (IN)

(21) Appl. No.: 18/688,418

(22) PCT Filed: Oct. 27, 2021
(86) PCT No.: PCT/IB2021/059807

§ 371 Date: Mar. 1, 2024

(30) Foreign Application Priority Data
Sep. 1, 2021 (IN) 202111039585

(51) Int. Cl. A61L 15/22; A61L 15/18; A61L 15/42; A61L 15/60; C08L 1/02; C08L 5/02; C08L 5/08

(52) U.S. Cl. CPC A61L 15/225; A61L 15/18; A61L 15/425; A61L 15/60; C08L 1/02; C08L 5/02; C08L 5/08; A61L 2400/04

(57) ABSTRACT

The present invention relates to the field of polymeric matrix for haemostatic application. In particular, the Invention provides an efficient polymeric haemostatic device/bandage with high swelling rate, enhanced mechanical property and high blood clotting ability.

### FIELD OF INVENTION

The present invention relates to the field of polymeric matrix for haemostatic application. In particular, the Invention provides an efficient polymeric haemostatic device/bandage with high swelling rate, enhanced mechanical property and high blood clotting ability.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art.

Most bleeding casualties arise within the first 30 minutes of the injury leading to high patient mortality which can be reduced by effective and especially with immediate action. Besides controlling bleeding quickly, haemostatic products that are ready-to-use, easy to apply for on-site responders in trauma situations, antiseptic and has high shelf life are generally accepted for forward care in the battle zone. Although, the advancement in material science has led to the development of various promising dressing, only a few made it to clinical translation.

In battlefields and trauma cases, uncontrolled haemorrhages and its associated severe complications remain one of the major factors in both defense and civilian deaths. From many decades, the mortality rate has increased due to continuous loss of blood which can be prevented by an ideal haemostatic dressing that forms a stable clot as well as stops excessive bleeding.

In the past, compression with gauze was a widespread practice for stopping blood loss in most injuries. Development of some new generation materials had already overcome some of the prevailing limitations, but lack of rapid coagulation and insufficient provision limited its more extensive application. These are also expensive, unable to curtail microbial infection and may lead to allergic reactions. The currently available haemostatic dressings (materials that prevent blood loss) are also not efficient enough to stop excessive bleeding.

A wide variety of materials are used at an individual level for haemostasis but fail in clinical use as they lack to fulfill all requirements. As elaborated above, many strategies for developing the haemostatic material have been devised using specific technologies like electrostatic spinning, emulsion polymerization, layer-by-layer self-impregnation, etc. The use of such methods has solved the problem of developing the composite materials to some extent; however, issues like flexibility, blood absorption rate, coagulation time, cost, incorporation of haemostatic agent, etc. are worth to mention which still persist. Many of such materials individually/composites have proven less effective, inadequate, expensive, associated with severe complications in in-vivo studies.

CN104208741A discloses adhesive bandage based on chitosan, oligochitosan, and enhancing polymer, wherein the chitosan and oligochitosan accounts for 85 to 95% of the total weight of the adhesive bandage, and the enhancing polymer is one or more of sodium alginate, glucan, hyaluronic acid polymer, and collagen.

U.S. Pat. No. 9,950,091B2 discloses composition based on a hydrogel matrix that includes at least one polymer cross linked, via ionic or covalent bonding, with both hyaluronic acid and alginic acid. The polymer is chitosan, poly L-Lysine, or a combination thereof.

However, the technologies disclosed in US '0091 and '741 are mainly based on chitin which is not efficient in stopping the heavy bleeding and there is still a need of efficient and better mechanisms and related product on the same.

U.S. Pat. No. 4,822,349 describes a non-bandage material used to treat bleeding. The material is sold by Z-Medica as “Quick-Clot” and is a granular form of zeolite, an aluminum silicate mineral. While use of this material may be preferable to bleeding to death, the attendant burning of tissue at and near the wound (and possible burn injury of medical personnel who are administering the material) is clearly a severe disadvantage. This side effect also reduces the ability of the material to be used for internal hemorrhage.

No perfect solution currently exists for the treatment of excessive or severe bleeding.

Many haemostatic products have been synthesized and are commercially available, presenting an approach of art as well as science to choose dressings over one another. However, the primary FDA approved haemostatic dressings like dry fibrin sealant dressing (DFSD), those based on collagen, chitosan, semcitite and zeolite (HemCon, QuikClot, WoundStat, etc) have significantly promoted rapid and effective hemostasis, but majority of them fail to fulfill the needed ideal requirements. The major drawbacks of these dressings are expensive, availability, non-antiseptic, and lack of rapid coagulation and may lead to allergic reactions. So, there is an immediate need to develop a dressing material which can have a high haemostatic ability and should protect the wound from secondary bacterial infections. It has also been observed that various haemostatic agents impregnated in the developed material or gauze leach-out into blood vessels, leading to severe vascular clots that may eventually become deadly.

Hence, there is a need of an efficient, cost effective and flexible bandage with high blood absorbing and antimicrobial capacity. There is also needed an efficient method of production of the flexible bandage with advantageous properties of quick blood clotting, high blood absorbing and antimicrobial capacity.

### OBJECT/S OF INVENTION

Primary object of the present invention is to overcome the limitation of prior art.

Another object of the present invention is to provide an efficient polymeric bandage for haemostatic application.

Another object of the present invention is to provide an efficient polymeric haemostatic device/bandage with high swelling rate, enhanced mechanical property, enhanced absorption rate and high blood clotting ability.

Another object of the present invention is to provide an efficient polymeric haemostatic device/bandage based on naturally derived polymers comprising chitosan and dextran.

Another object of the present invention is to provide an efficient polymeric haemostatic device/bandage based on cryogelled polymers.

Another object of the present invention is to provide a cryogel composition with high absorption rate and rapid blood clotting ability.

Another object of the present invention is to provide a cost effective bandage with prolonged shelf life in lyophilized form.

Another object of the present invention is to provide a method, for preparing the cryogel composition, which combines biocompatible and haemocompatible polymer-based materials with other topical haemostatic agents.

Another object of the present invention is to provide a method based on cryogelation for obtaining the polymeric bandage for haemostatic application.

### SUMMARY OF THE INVENTION

In an aspect, there is provided a cryogel composition for a hemostatic bandage comprising:
a) Chitosan (C)-O-Dex (D)-Kaolin (K)-CaCl₂ (Ca) composite (CDKCa)
b) Topical agents comprising Kaolin, calcium chloride
   where said dextran is oxidized and cryogelled at the sub-zero temperature;
   where said composition comprises approx. Ig chitosan; 2.5 g of Kaolin, 0.5 g of CaCl₂), approx. 3 g of oxidised dextran.

In another aspect, there is provided a method of surface modification of dextran which comprises oxidation of hydroxyl groups at adjacent carbons of glucose units by sodium periodate (NaIO₄) leading to the formation of two aldehydic groups per glucose unit due to the cleavage of bond between the adjacent carbons containing hydroxyl groups and the formation of multifunctional dextran with high reactive groups for coupling with molecules having amines.

In another aspect of the Invention, there is provided a process of preparing the composition as described above, comprising the steps of:
a) Fabricating the Chitosan (C)-O-Dex (D)-Kaolin (K)-CaCl₂ (Ca) composites (CDKCa) by the steps of dissolving chitosan (1 g) in 60 ml of acetic acid (1%) for 4-6 h;
b) Adding 2.5 g of Kaolin and 0.5 g of CaCl₂ in 15 ml of mQ-water followed by proper mixing to obtain a solution;
c) Adding chitosan solution with the solution obtained in step (b) followed by maintaining it on rocker to allow complete homogenization followed by cooling at 4° C. for half an hour
d) Obtaining solution by dissolving 3 g of oxidised dextran in 25 ml of mQ-water at 70° C. with continuous stirring for 30 min. followed by cooling at 4° C. for half an hour
e) Adding the dextran solution obtained in step (d) to the solution obtained in step (c) followed by pouring of the final solution in the desired mould having thin cotton membrane;

In another aspect, there is provided a therapeutic bandage comprising a base layer embedded with a polymeric matrix of cryogel composition as described above, wherein said cryogel composition comprises:
c) Chitosan (C)-O-Dex (D)-Kaolin (K)-CaCl₂ (Ca) composites (CDKCa)
d) Topical agents comprising Kaolin, calcium chloride
   where said dextran is oxidized and cryogelled at the sub-zero temperature;
   where said composition comprises approx. Ig chitosan; 2.5 g of Kaolin, 0.5 g of CaCl₂), approx. 3 g of oxidised dextran.

### DETAILED DESCRIPTION OF DRAWING

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings in which:

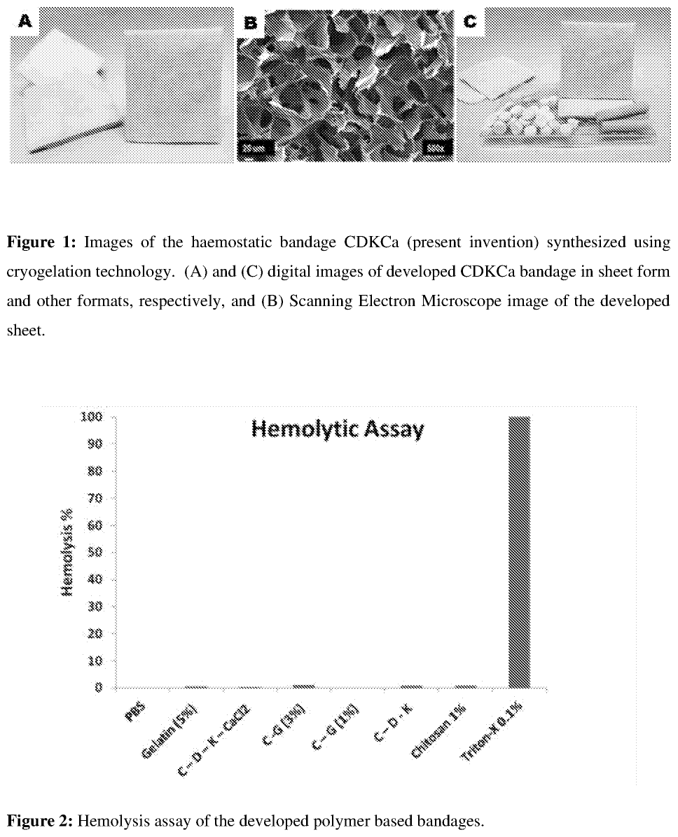

FIG. 1: illustrate images of the haemostatic bandage CDKCa (the present invention) synthesized using cryogelation technology. (A) and (C) digital images of developed CDKCa bandage in sheet form and other formats, respectively, and (B) Scanning Electron Microscope image of the developed sheet.

FIG. 2: illustrate hemolysis assay of the developed polymer based bandages.

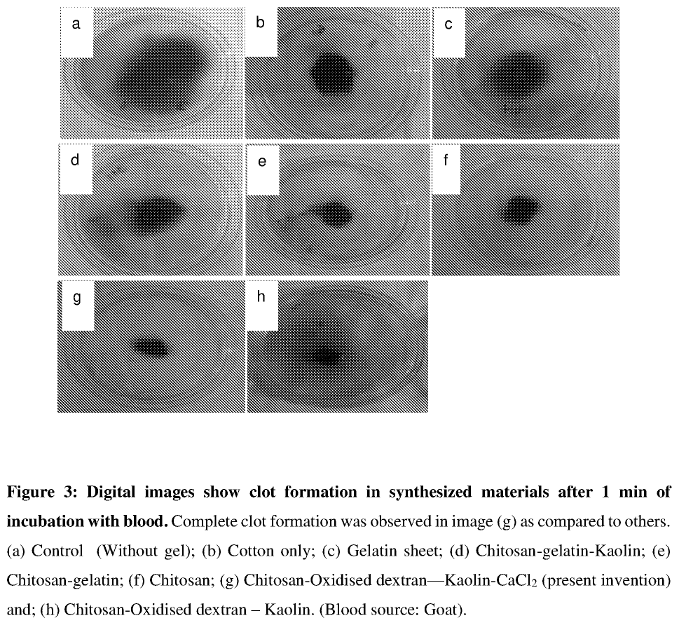

FIG. 3: illustrate Digital images show clot formation in synthesized materials after 1 min of incubation with blood. Complete clot formation was observed in image (g) as compared to others. (a) Control (Without gel); (b) Cotton only; (c) Gelatin sheet; (d) Chitosan-gelatin-Kaolin; (e) Chitosan-gelatin; (f) Chitosan; (g) Chitosan-Oxidised dextran-Kaolin-CaCl₂ (the present invention) and; (h) Chitosan-Oxidised dextran-Kaolin. (Blood source: Goat).

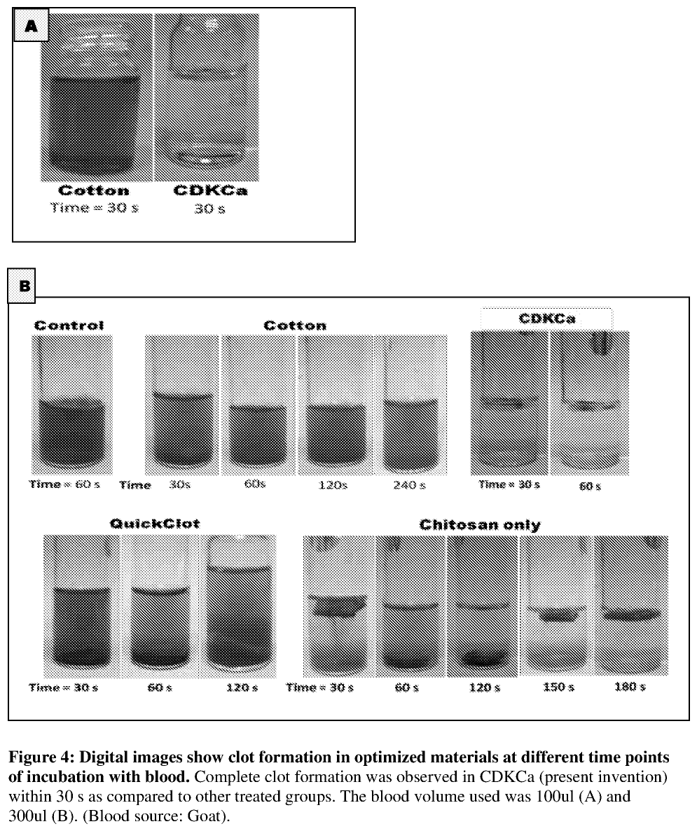

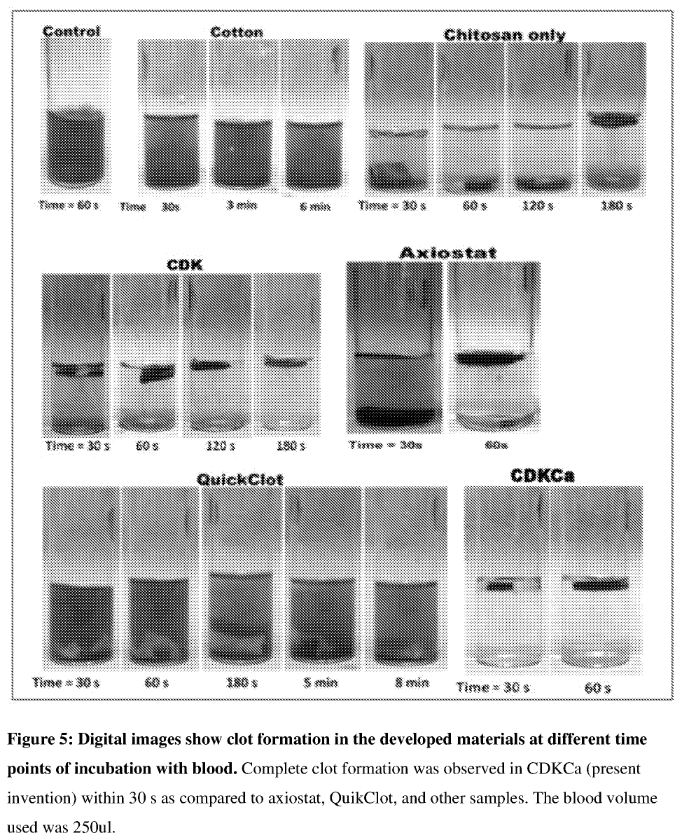

FIG. 4: illustrate Digital images show clot formation in optimized materials at different time points of incubation with blood. Complete clot formation was observed in CDKCa (the present invention) within 30 s as compared to other treated groups. The blood volume used was 100 ul (A) and 300 ul (B). (Blood source: Goat).

FIG. 5: illustrate Digital images show clot formation in the developed materials at different time points of incubation with blood. Complete clot formation was observed in CDKCa (the present invention) within 30 s as compared to axiostat, QuikClot, and other samples. The blood volume used was 250 ul.

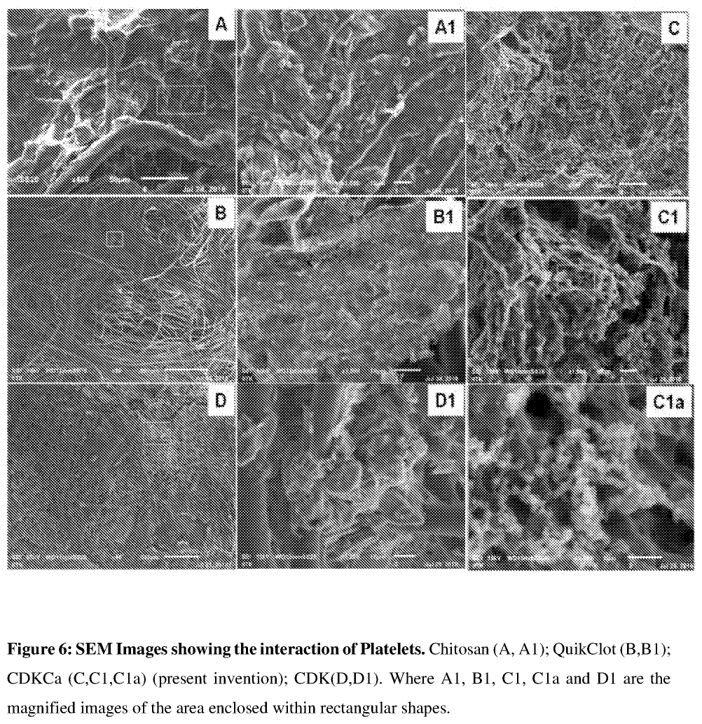

FIG. 6: illustrate SEM Images showing the interaction of Platelets. Chitosan (A, A1); QuikClot (B, B1); CDKCa (the present invention) (C, C1, C1a); CDK (D, D1). Where A1, B1, C1, C1a and D1 are the magnified images of the area enclosed within rectangular shapes.

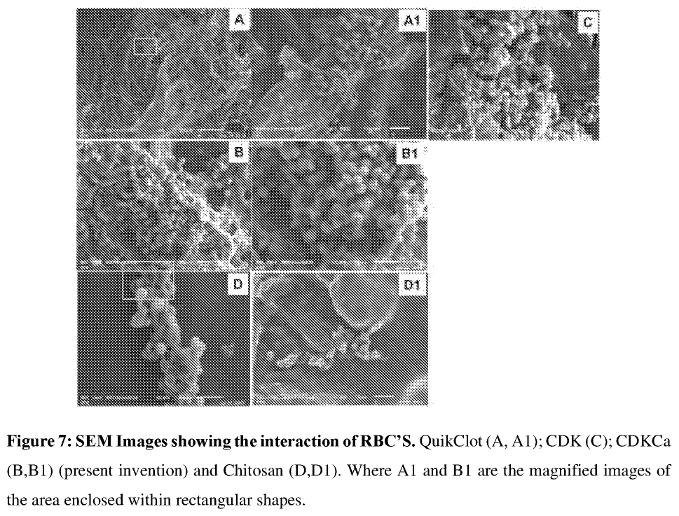

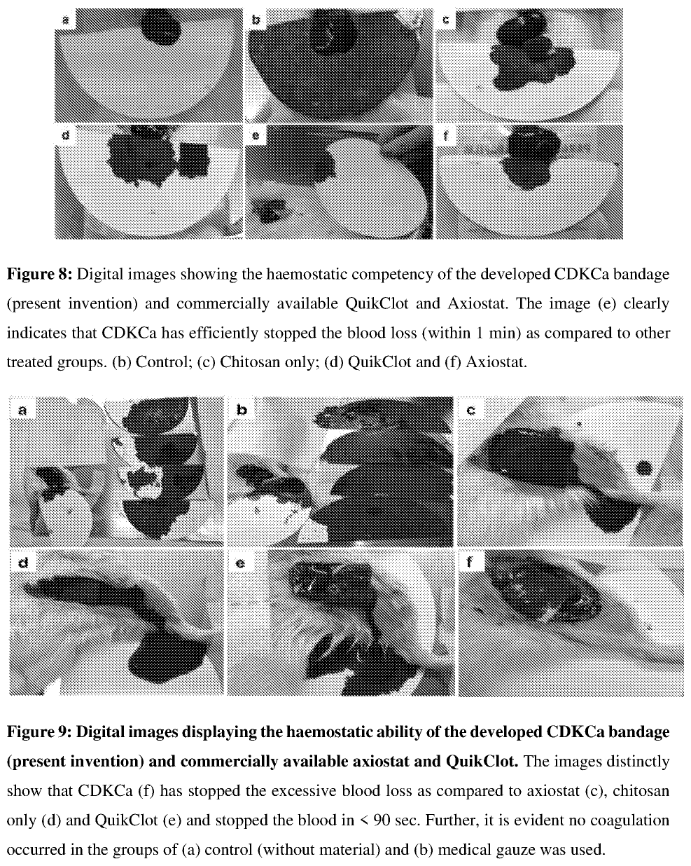

FIG. 7: illustrate SEM Images showing the interaction of RBC'S. QuikClot (A, A1); CDK (C); CDKCa (the present invention) (B, B1) and Chitosan (D, D1). Where A1 and B1 are the magnified images of the area enclosed within rectangular shapes FIG. 8: illustrate Digital images showing the haemostatic competency of the developed CDKCa bandage (the present invention) and commercially available QuikClot and Axiostat. The image (e) clearly indicates that CDKCa has efficiently stopped the blood loss (within 1 min) as compared to other treated groups. (b) Control; (c) Chitosan only; (d) QuikClot and (f) Axiostat.

FIG. 9: illustrate Digital images displaying the haemostatic ability of the developed CDKCa bandage (the present invention) and commercially available axiostat and QuikClot. The images distinctly show that CDKCa (f) has stopped the excessive blood loss as compared to axiostat (c), chitosan only (d) and QuikClot (e) and stopped the blood in <90 sec. Further, it is evident no coagulation occurred in the groups of (a) control (without material) and (b) medical gauze was used.

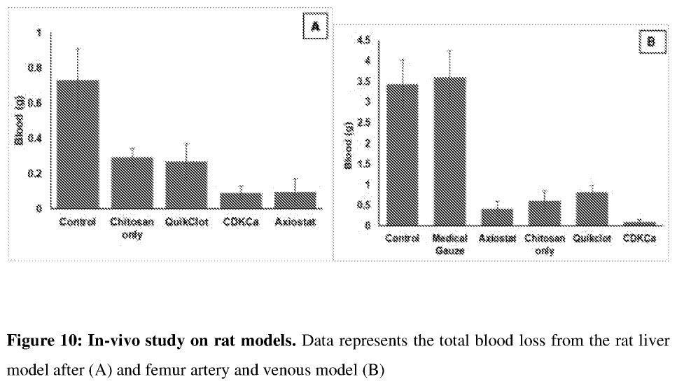

FIG. 10: illustrate In-vivo study on rat models. Data represents the total blood loss from the rat liver model after (A) and femur artery and venous model (B)

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof. Throughout the patent specification, a convention employed is that in the appended drawings, like numerals denote like components.

The present invention provides a cryogel composition and highly efficient polymeric bandage based on the same for haemostatic application especially during excessive bleeding in case of battlefields and civilian trauma. The invention is efficient in stopping excessive blood loss and will be useful to common public and defense people. The bandage will act as a first-hand emergency bandage in critical conditions (war, trauma), where seconds matter to save a precious life.

The Invention provides an efficient polymeric haemostatic device/bandage with high swelling rate, enhanced mechanical property and high blood clotting ability. This polymeric bandage works synergistically to maintain haemostasis by forming a stable clot, besides accelerating the clot formation. The bandage has high blood absorption rate besides efficient in rapid clotting. The haemostatic device/Bandage is based on cryogelation technology and a new composition to stop excessive bleeding, specifically critical and deadly bleeding in trauma care and battlefields. The cryogelation will help to provide a well interconnected macroporous network within the bandage, necessary for rapid fluid uptake and high swelling rate. The use of cryogelation technology further ensures proper integration of multiple haemostatic agents into the bandage for efficient and rapid clotting of blood. In addition, the technology used for fabrication of this invention substantially stopped the leaching of haemostatic agents from the material during practical application thereby avoiding any chances of risks due to systemic thrombosis and emboli formation. The in-vitro and in-vivo evaluation of the developed haemostatic bandage showed best results with rapid blood coagulation and good antiseptic properties.

The cryogel composite showed high absorption rate and rapid blood clotting, an important property of an ideal haemostatic dressing. The bandage is flexible, ready to use that can be cut in various sizes for distinct wounds and has high shelf life.

In an embodiment, the composite haemostatic bandage prepared from polymeric materials i.e. chitosan and oxidized dextran incorporated with other topical agents (kaolin and calcium chloride) using cryogelation technology. The chitosan based bandage not only controls bleeding quickly but also have a high blood uptake capacity. This helps to eliminate the need for frequent changing of the dressing. The bandage has optimum swelling and mechanical properties, which is enhanced further by incorporation of cotton mesh. Further, the bandage is antiseptic due to the inherent antimicrobial property of the incorporated polymer and has a high shelf life. The developed haemostatic matrices are able to stop bleeding quickly without having any issues of biocompatibility.

In an embodiment, the bandage comprises a cotton mesh incorporated within the polymeric cryogel, that enhances its mechanical stability and flexibility along with controlling the swelling of the bandage. Besides, being able to stop bleeding quickly, the chitosan-based composite is non-cytotoxic and inexpensive as compared to other available products.

The composite haemostatic bandage is prepared from chitosan and tailored the properties of dextran, incorporated with kaolin and calcium chloride as the potent haemostatic agents. The Invention uses specific cryogelation technologies, to combine the biocompatible and haemocompatible polymer-based materials with other topical haemostatic agents, which can directly activate the coagulation cascade to improve the haemostatic potential. The method of fabrication has led to an interconnected porous structure with optimum pore size, high fluid absorption rate, and very rapid blood clot formation.

These advantageous properties of the invention directly depends on certain major fabrication steps, mainly the appropriate concentration of polymers and haemostatic agents besides using a desired crosslinking agent. The porous interconnectivity as well as the mechanical property is highly dependent on the concentration of ingredients and temperature of fabrication. To have a desired bandage for ideal haemostatic applications, time of incubation during cryogelation also plays a critical role. The claimed invention has customized these parameters to achieve the best result.

In an embodiment, the bandage is based on polymers (chitosan and oxidized dextran) and haemostatic agents (comprising kaolin and calcium chloride) in the cryogel bandage. Kaolin, a natural clay consists of a network of aluminosilicate units, giving a net negative surface charge that plays a significant role mainly in activating the factor XI and XII thereby formation of fibrin. Calcium ions play a critical role in blood clotting, they are important in the activation of many clotting factors of multistep coagulation cascade as well as are involved in the formation of fibrin clot by triggering the production of thrombin.

In an embodiment, the Invention provides a polymeric bandage based on non-limiting biocompatible materials comprising chitosan (C) and dextran (D). The biocompatible materials are cryogelled at the sub-zero temperature using cryogelation technology.

In an embodiment, the cryogel composition for a hemostatic bandage comprises:
a) Chitosan (C)-O-Dex (D)-Kaolin (K)-CaCl₂ (Ca) composite (CDKCa)
b) Topical agents comprising Kaolin, calcium chloride
   where said dextran is oxidized and cryogelled at the sub-zero temperature.

In an embodiment, the composition comprises approx. Ig chitosan, 2.5 g of Kaolin, 0.5 g of CaCl₂, approx. 3 g of oxidised dextran.

In an embodiment, the dextran is oxidized through the surface modification.

In an embodiment, the surface modification of dextran comprises oxidation of hydroxyl groups at adjacent carbons of glucose units by sodium periodate (NaIO4) leading to the formation of two aldehydic groups per glucose unit due to the cleavage of bond between the adjacent carbons containing hydroxyl groups and the formation of multifunctional dextran with high reactive groups for coupling with molecules having amines.

In an embodiment, the dextran is partially oxidized in the presence of an aqueous solution of sodium periodate by the steps of:
a) adding an aqueous solution of sodium periodate, comprising 0.5 g of sodium periodate dissolved in 2.5 ml water, drop wise to dextran solution followed by vigorous stirring at 4° C. for 1 h in the dark at 300 rpm followed by addition of approx. 1 ml ethylene glycol to stop the reaction by consuming the unused sodium periodate;
b) dialyzing the reaction mixture obtained from step (a) at 4-8° C. against milli-Q H₂O for approx. 3 days with regular change of milli-Q H₂O every 6 hour;
c) collecting the dialyzed solution and freeze-drying to obtain oxidized dextran (ODex) for desired use.

In an embodiment, the dextran is partially oxidized in the presence of an aqueous solution of sodium periodate by the steps of:
One gram of dextran (70 kDa) was properly dissolved in 10 ml of milli-Q H₂O and was subjected to partial oxidation with sodium periodate to generate aldehyde functionalities. Briefly, an aqueous solution of sodium periodate (0.5 g, dissolved in 2.5 ml water) was drop wise added to dextran solution. The solution was vigorously stirred at 4° C. for 1 h in the dark at 300 rpm followed by the addition of ethylene glycol (1 ml) to stop the reaction by consuming the unused sodium periodate. The reaction mixture at 4-8° C. was dialyzed extensively against milli-Q H₂O for 3 days with regular change of milli-Q H₂O every 6 h. Lastly, the dialyzed solution was collected and freeze-dried to obtain oxidized dextran (ODex) for desired use. The estimated degree of substitution was found approx. 37%.

In an embodiment, there is provided a process of preparing the composition as described above. The process comprises the steps of:
a) Fabricating the Chitosan (C)-O-Dex (D)-Kaolin (K)-CaCl₂ (Ca) composites (CDKCa) by the steps of dissolving chitosan (1 g) in 60 ml of acetic acid (1%) for 4-6 h;
b) Adding 2.5 g of Kaolin and 0.5 g of CaCl₂ in 15 ml of mQ-water followed by mixing to obtain a solution;
c) Adding chitosan solution to the solution obtained in step (b) followed by maintaining it on rocker to allow complete homogenization followed by cooling at 4° C. for half an hour.
d) Obtaining solution by dissolving 3 g of oxidised dextran in 25 ml of mQ-water at 70° C. with continuous stirring for 30 min. followed by cooling at 4° C. for half an hour.
e) Adding the dextran solution obtained in step (d) to the solution obtained in step (c) followed by pouring of the final solution in the desired mould having thin cotton membrane.

In an embodiment, the therapeutic bandage comprising a base layer embedded with a polymeric matrix of cryogel composition as described above. The cryogel composition comprises:
a) Chitosan (C)-O-Dex (D)-Kaolin (K)-CaCl₂ (Ca) composites (CDKCa)
b) Topical agents comprising Kaolin and calcium chloride where said dextran is oxidized and cryogelled at the sub-zero temperature.

In an embodiment, the therapeutic bandage comprises approx. 1 g chitosan, 2.5 g of Kaolin, 0.5 g of CaCl₂, approx. 3 g of oxidised dextran.

In an embodiment, the base layer is made of material comprising cotton or material with alike properties.

In an embodiment, the bandage is prepared by following steps:
a) Fabricating the Chitosan (C)-O-Dex (D)-Kaolin (K)-CaCl₂ (Ca) composites (CDKCa) by the steps of dissolving chitosan (1 g) in 60 ml of acetic acid (1%) for 4-6 h;
b) Adding 2.5 g of Kaolin and 0.5 g of CaCl₂ in 15 ml of mQ-water followed by mixing to obtain a solution;
c) Adding chitosan solution to the solution obtained in step (b) followed by maintaining it on rocker to allow complete homogenization followed by cooling at 4° C. for half an hour.
d) Obtaining solution by dissolving 3 g of oxidised dextran in 25 ml of mQ-water at 70° C. with continuous stirring for 30 min. followed by cooling at 4° C. for half an hour.
e) Adding the dextran solution obtained in step (d) to the solution obtained in step (c) followed by pouring of the final solution in the desired mould having thin cotton membrane;
f) Cryogelating of the mould resulting from step (e) by incubating in a cryostat at −12° C. for 14 h. to obtain the synthesized cryogel columns/sheets which were then given freeze thaw cycles to remove all the unreacted polymers and increase their strength followed by lyophilization for storage.

In an embodiment, the composite was fabricated by dissolving chitosan (1 g) in 60 ml of acetic acid (1%) for 4-6 h. Next, 2.5 g of Kaolin and 0.5 g of CaCl₂ were mixed in 15 ml of mQ-water. After proper mixing, the solution was added to the chitosan solution and was kept on rocker to allow complete homogenization. In addition, 3 g of oxidised dextran was dissolved in 25 ml of mQ-water at 70° C. with continuous stirring for 30 min. The two solutions were then pre-cooled separately at 4° C. for half an hour. After cooling of the solutions for desired time, the dextran solution was added to the other solution (containing chitosan, Kaolin and CaCl₂) and were mixed appropriately followed by pouring of the solution in the desired mould having thin cotton membrane. The mould was then allowed for cryogelation by keeping it in a cryostat at −12° C. for 14 h. The synthesized cryogel columns/sheets were then given freeze thaw cycles to remove all the unreacted polymers and increase their strength followed by lyophilization for their proper storage.

In an embodiment, the synthesis happens at sub-zero temperature comprising −12° C. for 14 h followed by freeze-thawing cycle. The optimized temperature of −12° C. for 14 h allows to have a desired crosslinking within the polymers, forming mechanically flexible and stable haemostatic bandage. The developed haemostatic bandage at sub-zero temperature resulted to the formation of macroporous structure with interconnected porous architecture that is essential for rapid and efficient fluid uptake without getting disintegrated or collapse during practical application. The freeze thaw cycle aids in achieving proper pore structure and in mechanical stability of the haemostatic bandage.

In order to make the bandage robust, proficient and cost effective, kaolin (K) and calcium chloride (Ca) as haemostatic agents were incorporated in the developed bandage that provides a synergistic effect in controlling excess bleeding. These components of fabricated matrix cause occlusion by protein precipitation or coagulation (cell entrapment), while others act on later steps of the clotting pathway thus initiating a biological response to stop bleeding. The polymer based cryogel bandage (CDKCa) is formed in various moulds to get appropriate shape for specific applications at distinct sites of injuries.

The physical properties of the developed haemostatic bandage were evaluated to examine its suitability for quick control of bleeding. The CDKCa haemostatic bandage showed very high absorption and swelling rate both in water and simulated body fluid. The property is very significant for an ideal haemostatic dressing and is attributed to the interconnectivity of pores in the developed cryogel sheet. Furthermore, CDKCa bandage showed rapid blood coagulation (in vitro) in the 30 s as compared to other chitosan based compositions as well as commercially available QuikClot. The CDKCa will come in contact with blood and thus was tested for haemolysis. The results showed the material is heamocompatible. Mechanical strength is a very important factor in a haemostatic dressing, and in the developed polymeric composite, it can be easily modulated by varying the concentration of the polymers or the temperature of synthesis. The fabricated CDKCa composite system and the bandage based on it exhibits a rapid and strong effect as a haemostatic dressing, which is highly efficient over the commercially available products.

### Advantageous Properties of the Bandage Provided by the Present Invention Over Existing Products:

The bandage showed quick blood coagulation because of its charge based polymers and stimulation of clotting cascade by topical agents.
The enhanced mechanical strength as compared to the basic chitosan based dressings increases the haemostatic or fluid absorption rate.
The bandage is cost effective and has prolonged shelf life in lyophilized form
The bandage is based on biocompatible, antibacterial and non-toxic polymers comprising chitosan and dextran and retains their functional properties till its use at the bleeding site.
The haemostatic bandage regulates the pore size and equilibrium swelling, which is important for its wide variety of application, at optimal polymeric concentration, crosslinking and temperature.

The surface modification of dextran comprises the oxidation of hydroxyl groups at adjacent carbons of glucose units by sodium periodate (NaIO₄). This reaction leads to the formation of two aldehydic groups per glucose unit due to the cleavage of bond between the adjacent carbons containing hydroxyl groups. The NaIO₄ based modification of dextran leads to the formation of multifunctional dextran with high reactive aldehydic groups for coupling with molecules having amines. In this invention the functionalized dextran forms Schiff base by reacting with amine groups present in the chitosan.

In an embodiment, the product is developed as a single unit in the form of cryogel patch/bandage using cryogelation process. The cryogelation technique makes the fabrication process easy and cost effective. The size and format of this can be easily modified depending on the desired application. The developed bandage has interconnected porous architecture leading to rapid fluid uptake as well as high swelling ability. The invention led to rapid blood clotting during excessive bleeding. The fabrication method of cryogelation does not any leaching of material ingredients from the bandage thereby preventing the risk of forming thrombosis or systemic emboli.

The Invention is further described with the help of non-limiting examples:

### Example 1

The haemostatic bandage comprises naturally derived polymers chitosan and dextran. The dextran is oxidized through the surface modification thereby allowing it to be used as a crosslinker as well as haemostatic agent due to its negative charge. The invention employs cryogelation technology in the fabrication of haemostatic bandage from hydrophilic polymers and eases while incorporating the haemostatic agents Kaolin and Ca²⁺ (from calcium chloride). The use of cryogelation technology not only helped to overcome the unwanted release of haemostatic agents at the injury site but also maintained the uniformity of fine porous structure, mimicking to the naturally occurring fibrin mesh. The porous network of the developed haemostatic device leads to fast swelling rate and entrapment of platelets and red blood cells thereby effectively reducing the coagulation time. In addition, the present invention provides a unique composition of the components in the form of developed haemostatic bandage with highly efficient and improved haemostasis. In particular, the composition of the inventive haemostatic bandage has been further embedded with the cotton membrane at the time of synthesis to present a flexible and mechanically robust bandage in order to assure its application at any unique geometrical shape and size.

Furthermore, this composition has led to the haemostatic bandage which is user friendly, less expensive, long shelf life, no exothermic reaction when applied. The cumulative and synergetic effect of these polymeric components i.e. chitosan and oxidized dextran with incorporated haemostatic agents i.e. Kaolin and Ca²⁺ in the developed material has shown the ability to rapidly form a stable fibrin clot. The clotting occurs due to surface charge interaction i.e. positive charge of chitosan with platelets and Ca²⁺ ions, a crucial role in activating clotting factors of the cascade, thrombin formation and finally role in the conversion of fibrinogen into fibrin. The haemostatic property is also efficiently improved by the negatively charged surface of oxidized dextran and kaolin through the stimulation of intrinsic coagulation cascade via autoactivation of clotting factor XII, which has a direct role in fibrin generation.

### Example 2

The fabricated dressings for haemostatic application have been evaluated for various physio-chemical properties as well as in both in-vitro and in-vivo studies. The experimental analysis was carried out and studied using specific samples.

Different types of polymer (chitosan)-based cryogel sheets that can be used as ideal haemostatic dressings were formed. These cryogel materials have been fabricated using different natural polymers and inorganic compounds. The materials used exhibit distinct advantages in stability, accessibility and cost efficiency with no biological toxicity or risk of disease transmission. Chitosan has been used as a haemostatic material due to its cationic and antimicrobial nature, besides being biocompatible in nature. Dextran is a hydrophilic polysaccharide and can easily be configured by altering its surface functionality. It has been used in the haemostatic application for both civilian and combat trauma. Oxidation of dextran will help both in crosslinking as well as in blood coagulation. So, initially, dextran was subjected to partial oxidation in the presence of an aqueous solution of sodium periodate to generate aldehyde functionalities. The generated aldehyde groups help in the crosslinking of chitosan in the fabricated bandages. The amount of aldehyde generated was calculated around 30%. The oxidized dextran was then used to fabricate the chitosan based cryogel sheets in different formats with and without haemostatic agents (Kaolin/Calcium chloride). Kaolin and Calcium chloride as a haemostatic agent has shown the efficacy in the stabilization of severe arterial hemorrhage and diffuse long-term bleeding from lethal liver injuries along with other materials. Kaolin also called as Chinese clay consists of aluminosilicate network that leads to the activation of blood coagulation cascade by stimulating the intrinsic pathway. Also, the negative charge on Kaolin surface triggers binding of factor XII, its autoactivation along with factor XI and their direct role in fibrin formation.

### Example 3

### Development and Characterization of Haemostatic Bandage

The approach towards the development of an ideal haemostatic dressing is very critical, and currently, cryogelation technology is one of the promising approaches for material fabrication. The cryogel sheets were developed using various polymers such as chitosan, gelatin, polyurethane, etc. with or without haemostatic agents during the optimization process. The various combinations of cryogel sheets developed, characterized and evaluated for blood clotting study were: (1) Chitosan (C)-O-Dex (D)-Kaolin (K)-CaCl₂) (Ca) composite (2) Gelatin Cryogel only (3) Chitosan-Gelatin (4) Chitosan only (5) Chitosan-O-Dex-Kaolin (6) Chitosan-Polyurethane (not formed). All the cryogel bandages were synthesized at sub-zero temperature (−12° C. for 14 hours) followed by freeze-thawing cycle, resulting in melting of the frozen solvent crystals, leaving behind a well interconnected porous network as confirmed by scanning electron microscopy as in FIG. 1B. The interconnectivity of pores is one of the key properties correlated to rapid fluid absorption rate, important for haemostatic dressings.

### Example 4

The bandage was made mechanically robust by embedding with cotton fibers to enhance its flexibility and mechanical property as in FIG. 1A. The fabrication technology of cryogelation has allowed to develop the bandage in various formats for multiple and distinctive application as haemostatic dressing, an advantage over others (FIG. 1C). The water and simulated body fluid (SBF) absorption/uptake of the fabricated sheets was studied and the results as in table 1, showed all the chitosan based cryogels sheets exhibited rapid and high fluid absorption rate, a property important for an ideal haemostatic dressing. The high absorption rate can be attributed to the interconnected porous structure of the cryogel.

TABLE 1
Fluid absorption/uptake of the haemostatic
sheets using cryogelation technology.

| | SBF absorption rate | |
|---|---|---|
| Sample | (400 ul) | Water uptake |
| Gelatin (5%) | 4.5 s | 85% in 10.34 s |
| CG (3%) | 6.30 s | 80% in 13 s |
| CG (1%) | 5.48 s | 85% in 11.2 s |
| CDK | 5.17 s | 90% in 11.33 s |
| CDKCa | 5.01 s | 85% in 10 s |
| Chitosan 1% | 5.14 s | 87% in 12 s |

### Example 5

Next, as a clotting indicative, the absorbance of haemoglobin (540 nm) was evaluated to test the haemostatic efficiency of developed composite bandage. Higher absorbance of haemoglobin indicates poor clotting while as low absorbance indicates better clotting. The developed bandage was examined for hemolytic analysis, carried out with separated human erythrocytes. Blood was drawn from volunteered human subjects by registered staff at the Department of Medicine and Transfusion, Govt. Medical College, Kanpur with blood samples were tested. Subjects were healthy adults ranging in age from 25 y to 30 y. Sodium citrate (3.8%) was used as an anticoagulant. The erythrocytes were separated as per the standard protocol, briefly; the samples were centrifuged at 1500 rpm for 10 min followed by the washing of pellet 3 times using 1× phosphate buffer saline (PBS, pH 7.2). The samples were incubated in 10 ml of blood: PBS (1:9) for 1 h at 37° C. and then measured at 540 nm post centrifugation. As shown in FIG. 2, PBS and all other polymeric samples do not show any haemolysis, except the Triton-x-100 (positive control) where 100% lysis was observed and in order to describe the hemolysis more directly, the positive control (Triton-x-100) was set as 100% of hemolysis. Thus, the developed materials exhibited better hemocompatibility and can be an ideal dressing for haemostatic application.

### Example 6

### Blood Clotting Assay:

As a functional assay, the developed materials were evaluated for blood coagulation test initially on goat blood (I and II) and later on human blood samples (III).
I. To analyze the effect of clotting, we collected a goat blood sample from local slaughter shop. During the optimization process, the blood clotting test on cryogel samples was done for a specific time (1 or 2 minutes) to form a clot from the blood. In case of control, the same volume of blood was left undisturbed on the plate. As shown in FIG. 3, in case of CDKCa composite (FIG. 3g) the clotting occurred within 1 min; however, no clotting was observed in control. The clotting time observed in other groups; CDK, chitosan only and chitosan-gelatin-kaolin was higher than 2 min. Also, in the control group, the clotting time was around 4.5 min. Thus, the results clearly indicated that the incorporation of kaolin and CaCl₂ during the fabrication of chitosan-ODex cryogel sheets led to increased and fast coagulation in comparison to control, cotton and gelatin sheet.
II. Based on our initial studies, the optimized cotton embedded CDKCa sheet was further evaluated along with commercially available QuikClot. In blood coagulation assay, it was observed that the rapid clot formation occurred in CDKCa composite i.e within 30 s for (anticoagulated whole blood) volume of 100 ul as well as when increased to 300 ul as compared to commercially available QuikClot gauze, chitosan cryogel and control as shown in FIGS. 4A and B. The leakage of red blood cells in the later treated groups could be observed even after above 3 min of incubation. These red blood cells that were not entrapped in the resultant clot undergo hemolysis on the addition of water. The experiment was carried at two different blood volumes 100 ul and 300 ul as in FIGS. 4 A and B, respectively.
III. Blood was drawn from volunteered human subjects by registered staff at the Department of Medicine and Transfusion, Govt. Medical College, Kanpur. Subjects were healthy adults ranging in age from 25 y to 30 y. The developed materials were further tested as mentioned above, for their clotting efficiency using anticoagulated human blood samples (250 ul) in-vitro. The samples were allowed to interact with blood for specific time followed by addition of 10 ml of deionized water to cease the reaction and dissolve the hemoglobin of uncoagulated cells. The results showed that CDKCa composite demonstrated high and rapid clotting potential (30 s) followed by axiostat (60 s) than other treated groups; CDK (180 s), QuikClot and pure chitosan (FIG. 5). The chitosan only group showed slight clotting ability but was not able to form a stable clot in order to retain the entrapped cells even after 3 min. Moreover, the commercially available QuikClot showed very poor clotting as well as no clotting was observed in pure cotton group. The fast and strong clot formation in CDKCa was due to the synergistic effect of positively charged polymers, incorporated kaolin and Ca²⁺ ions and their significant role in activating clotting cascade [18 and 19].

### Example 7

### Effect of Material Interface on Cell Interaction:

The influence of the developed CDKCa on blood clotting and to further observe the mechanism of heamostasis were evaluated by the effect of material interface on the adhesion of the platelets and red blood cells (RBC'S). The blood cells adhered on the material surface as observed by SEM images (FIGS. 6 and 7). The initial step of forming a plug to cease blood is contributed by platelets, and the results indicated very large platelet adhesion and aggregation in the developed CDKCa sheet followed by CDK sheet as shown in (FIGS. 6C and D). The amount of adhered platelets was less in pure chitosan sheet, and a few were observed in commercially available QuikClot gauze (FIGS. 6A and B). The results thus clearly confirmed the high ability of CDKCa sheet to stimulate the aggregation and adhesion of platelet as compared to other materials as well as QuikClot. The presence of pseudopods and irregularity in the shape of platelets adhered to the surface of the developed sheet can be attributed to their active form [20]. Moreover, incubation of erythrocytes further demonstrated a large amount of RBC'S gathered on the interface of CDKCa bandage as compared to other materials as seen in (FIG. 7B), besides maintaining their distinctive morphology. In comparison to CDKCa bandage, fewer erythrocyte aggregates were observed in the CDK sheet (FIG. 7C), followed chitosan sheet (FIG. 7D) and sparsed number in case of QuikClot gauze (FIG. 7A). In addition to the presence of a very high number of platelets and RBC's on the surface of developed CDKCa sheet in comparison to other materials, a vast fibrin mesh network has entrapped the cells to coalesce them into a clot. The results thus were well accordant to the above-mentioned whole blood clotting assay and further may be ascribed to porosity, high swelling rate, the cumulative effect of charge-based interaction of polymers and incorporated haemostatic agents of the developed bandage [14, 15, 17, 19].

### Example 8

### Blood Coagulation in an SD Rat Models:

The animal studies were carried out on Sprague-Dawley (SD) rats (250-300 g) which were directed in accordance with the “Guide for the Care and Use of Laboratory Animals.” All the in-vivo experimental protocols were approved by the Indian Animal Ethical Committee of Indian Institute of Technology Kanpur—IAEC IITK (Protocol No. ITK/IAEC/2017/1061).

The animals anesthetized using 2.5% isoflurane along with 21% of oxygen support were shaved and sanitized by 70% ethanol at desired sites, and then the rats were fixed on Styrofoam board, tilted at 45 degrees approx. The Axiostat and QuikClot, commercially available FDA approved products, were used as a standard haemostatic dressing for evaluating our developed CDKCa bandage for haemostatic application.

### Example 9

### Blood Coagulation in SD Rat Liver Model:

The rats were divided into 5 groups (No treatment group: control; Treated groups: chitosan only, QuikClot, CDKCa, and Axiostat) with 6 animals per group. Briefly, the 5 mm deep liver puncture model was created by the 20-G needle on the left lateral liver lobe exposed by abdominal incision. A pre-weighed filter paper was kept under the liver lobe, and blood flow from the injury was allowed freely for 5 s before applying the dressings. The evaluation of haemostatic effect by the treatment groups was evaluated by bleeding time and determining the difference in the weight of filter paper used within the respective groups after a liver puncture (FIG. 8). The results showed that bleeding continued for more than 5 min in control as compared to the treated groups (FIG. 8b). In treated groups, chitosan only and QuikClot (Commercially available FDA approved dressing) groups showed some improvement in clotting time 2.5 min and 3.47 min respectively, but not as significant as observed in CDKCa and axiostat treated group (FIGS. 8c and d). The CDKCa group has highly reduced clotting time to ˜57 s and the amount of bleeding as evident from the filter paper used in the group (FIG. 8e). The decrease in clotting time ˜65 s was observed in the axiostat group (FDA approved), apparent from presence of blood on the filter paper in comparison to control, chitosan, and QuikClot groups (FIG. 8f). In addition, the haemostatic outcome was confirmed by determining the amount of blood loss from each group as shown in (FIG. 10a). The total bleeding in the control (untreated group) was approx. 733 mg followed by treated groups chitosan and QuikClot with a reduction in bleeding to −294 mg and 271 mg, respectively. Treated groups CDKCa and axiostat (˜97 mg) exhibited substantial reduction in bleeding among all other groups, especially CDKCa, which showed the significantly rapid blood clotting and bleeding being reduced to less than 81 mg.

### Example 10

### Blood Coagulation in SD Rat Femur Artery and Venous Model:

The model was developed to mimic the field challenges of bleeding during trauma and battlefield. Also, initially bleeding from injured artery predominates but later venous bleeding becomes more appropriate due to mean pressure drop in the artery. The study of this femur model involves 6 treatment groups with 6 animals in each group. The 6 groups include control (no treatment group), gauze only, axiostat, QuikClot, chitosan only and CDKCa. All the animals were anesthetized and sterilized before any surgical procedure as mentioned in the above section. The femoral vein and artery and were exposed after making an incision in the left leg, and these exposed vessels were cut using a scalpel. A pre-weighed filter paper was immediately put under the injured leg to absorb the blood from the cut vessels prior to application of the bandage at the site. The total amount of blood loss accumulated on the filter paper and time to stop bleeding was recorded for efficient evaluation of haemostasis by the treating groups. The results of CDKCa group displayed rapid blood coagulation and a significant reduction in clotting time in comparison to other treated groups. In CDKCa group, the clotting was achieved quickly within 92 s without any further flow of blood to the filter paper (FIG. 9f). The other groups with some slight clotting efficiency were observed in axiostat, chitosan only and QuikClot only with clotting time of 197 s, 237 s, and 273 s, respectively besides the continuous flow of blood from the dressing was observed as absorbed on the filter paper (FIG. 9c-e). In addition, gauze only and the control group did not show any clotting property, and bleeding continued for more than 7 min that was entirely absorbed on several filter papers (FIGS. 9a and b). The results were also confirmed from the evaluation of total blood loss in the control as well as other treated groups, presented in (FIG. 10b). The extensive and continuous bleeding was observed both in control and medical gauze group, determined to be 3.4 g and 3.6 g, respectively as the total blood loss from the injury. In contrast to control, the total amount of bleeding in other treated groups was substantially reduced but not as statistically significant as in CDKCa treated group which showed stable and rapid clotting, reducing bleeding to 92 mg approximately. The reduction in bleeding by treated groups like axiostat, chitosan only and QuikClot were 420 mg, 625 mg, and 823 mg respectively, showing moderate haemostasis as compared to CDKCa group. Hence, the above discussed experimental study demonstrated that the rapid and stable clotting efficiency was exhibited by the developed CDKCa bandage and could be due to the synergistic activity of the specific polymers and incorporated elements. Further, the high blood absorption property of the bandage has led to increased cell aggregation and formation of a stable clot.

We claim:

1. A cryogel composition for a hemostatic bandage comprising:
a) Chitosan (C)-O-Dex (D)-Kaolin (K)-CaCl₂ (Ca) composite (CDKCa)
b) Topical agents comprising Kaolin, calcium chloride
where said dextran is oxidized and cryogelled at the sub-zero temperature;
where said composition comprises approx. 1 g chitosan; 2.5 g of Kaolin, 0.5 g of CaCl₂, approx. 3 g of oxidised dextran.

2. The composition as claimed in claim 1, wherein said dextran is oxidized through the surface modification.

3. The composition as claimed in claim 1, wherein said surface modification of dextran comprises oxidation of hydroxyl groups at adjacent carbons of glucose units by sodium periodate (NaIO₄) leading to the formation of two aldehydic groups per glucose unit due to the cleavage of bond between the adjacent carbons containing hydroxyl groups and the formation of multifunctional dextran with high reactive groups for coupling with molecules having amines.

4. The composition as claimed in claim 1, wherein dextran is partially oxidized in the presence of an aqueous solution of sodium periodate by the steps of:
a) adding an aqueous solution of sodium periodate, comprising 0.5 g of sodium periodate dissolved in 2.5 ml water, drop wise to dextran solution followed by vigorous stirring at 4° C. for 1 h in the dark at 300 rpm and addition of approx. 1 ml ethylene glycol to stop the reaction by consuming the unused sodium periodate;
b) dialyzing the reaction mixture obtained from step (a) at 4-8° C. against milli-Q H₂O for approx. 3 days with regular change of milli-Q H₂O every 6 h.
c) collecting the dialyzed solution and freeze-drying to obtain oxidized dextran (ODex) for desired use.

5. A process of preparing the composition as claimed in claim 1 comprising:
a) Fabricating the Chitosan (C)-O-Dex (D)-Kaolin (K)-CaCl₂ (Ca) composites (CDKCa) by the steps of dissolving chitosan (1 g) in 60 ml of acetic acid (1%) for 4-6 h;
b) Adding 2.5 g of Kaolin and 0.5 g of CaCl₂ in 15 ml of mQ-water followed by mixing to obtain a solution;
c) Adding chitosan solution to the solution obtained in step (b) followed by maintaining it on rocker to allow complete homogenization followed by cooling at 4° C. for half an hour.
d) Obtaining solution by dissolving 3 g of oxidised dextran in 25 ml of mQ-water at 70° C. with continuous stirring for 30 min. followed by cooling at 4° C. for half an hour.
e) Adding the dextran solution obtained in step (d) to the solution obtained in step (c) followed by pouring of the final solution in the desired mould having thin cotton membrane.

6. A therapeutic bandage comprising a base layer embedded with a polymeric matrix of cryogel composition as claimed in claim 1, wherein said cryogel composition comprises:
a) Chitosan (C)-O-Dex (D)-Kaolin (K)-CaCl₂ (Ca) composites (CDKCa)
b) Topical agents comprising Kaolin, calcium chloride
where said dextran is oxidized and cryogelled at the sub-zero temperature;
wherein said composition comprises approx. 1 g chitosan; 2.5 g of Kaolin, 0.5 g of CaCl₂, approx. 3 g of oxidised dextran.

7. The bandage as claimed in claim 6, wherein said base layer is made of material comprising cotton or material with alike properties.

8. A process of preparing the bandage as claimed in claim 1, comprising:
a) Fabricating the Chitosan (C)-O-Dex (D)-Kaolin (K)-CaCl₂ (Ca) composites (CDKCa) by the steps of dissolving chitosan (1 g) in 60 ml of acetic acid (1%) for 4-6 h;
b) Adding 2.5 g of Kaolin and 0.5 g of CaCl₂ in 15 ml of mQ-water followed by mixing to obtain a solution;
c) Adding chitosan solution to the solution obtained in step (b) followed by maintaining it on rocker to allow complete homogenization followed by cooling at 4° C. for half an hour
d) Obtaining solution by dissolving 3 g of oxidised dextran in 25 ml of mQ-water at 70° C. with continuous stirring for 30 min. followed by cooling at 4° C. for half an hour
e) Adding the dextran solution obtained in step (d) to the solution obtained in step (c) followed by pouring of the final solution in the desired mould having thin cotton membrane;
f) Cryogelating the mould resulting from step (e) by incubating in a cryostat at −12° C. for 14 h. to obtain the synthesized cryogel columns/sheets which were then given freeze thaw cycles to remove all the unreacted polymers and increase their strength followed by lyophilization for storage.
